(19) United States
(12) Patent Application Publication
(10) Pub. No.: US 2011/0295107 A1
(43) Pub. Date: Dec. 1, 2011

(54) Catheter System for Measuring and Marking Vessel Characteristics

(75) Inventors: Soroosh Kargar, Lake in the Hills, IL (US); Weng Lei, Mount Prospect, IL (US)

(73) Assignee: SIEMENS MEDICAL SOLUTIONS USA, INC., Malvern, PA (US)

(21) Appl. No.: 13/022,645

(22) Filed: Feb. 8, 2011

Related U.S. Application Data
(60) Provisional application No. 61/347,875, filed on May 25, 2010.

(51) Int. Cl. A61B 5/107; A61B 5/05

(52) U.S. Cl. 600/424; 600/481

(57) ABSTRACT

A system marks and measures a Vessel lumen, heart valve or body cavity using a circumference measuring catheter. A catheter system comprises a catheter device including a loop having an adjustable circumference that is adjustable within a vessel. The catheter device includes a means for adjusting the circumference of the loop and a means for measuring adjusted loop circumference. An output unit sends data indicating the measured loop (vessel) circumference or cross-sectional area derived using the measured loop circumference for presentation to a user.

This is a non-provisional application of provisional application serial No. 61/347,875 filed May 25, 2010, by S. Kargar et al.

### FIELD OF THE INVENTION

This invention concerns a catheter system comprising a catheter device including, a loop having an adjustable circumference and being adjustable within a vessel for measuring vessel internal circumference or vessel internal cross-sectional area.

### BACKGROUND OF THE INVENTION

Known systems for determining vessel stenosis have limited accuracy. Known systems are also unable to measure stenosis of vessels using images in cases where serious anomalies, foreshortening or angles of an image of a vessel render the stenosis obscure. In these cases, it is also not possible for experienced physicians to estimate the degree of Stenosis. A system according to invention principles addresses these deficiencies and related problems.

### SUMMARY OF THE INVENTION

A system marks and measures a Vessel lumen, heart valve or body cavity using a circumference measuring catheter. A catheter system comprises a catheter device including a loop having an adjustable circumference that is adjustable within a vessel. The catheter device includes a means for adjusting the circumference of the loop and a means for measuring adjusted loop circumference. An output unit sends data indicating the measured loop (vessel) circumference or cross-sectional area derived using the measured loop circumference for presentation to a user.

### BRIEF DESCRIPTION OF THE DRAWING

FIG. 1 shows a known catheter used in a hospital, for example.

FIG. 2 shows a catheter system for measuring vessel internal circumference, according to invention principles.

FIG. 3 shows a catheter system for measuring vessel internal circumference, according to invention principles.

FIG. 4 shows a catheter device including an automatic motorized unit for measuring vessel internal circumference, according to invention principles.

FIG. 5 shows a flowchart of a process used by a catheter system for measuring vessel internal circumference, according to invention principles.

### DETAILED DESCRIPTION OF THE INVENTION

A catheter system according to invention principles advantageously incorporates a measuring loop at the tip of the catheter. The system controls the size of the measuring loop that is located at the tip of the catheter and in one embodiment a device similar to a micrometer is incorporated in the catheter and used in measuring length of loop employed and loop circumference. The result of the loop size is displayed on a control consul workstation. A control mechanism increases and decreases the size of the loop at the tip of the catheter that is placed proximal to the Stenosis. In one embodiment a micrometer is used to measure loop circumference increase and decrease and in another embodiment a motorized unit automatically adjusts loop circumference to the size of a vessel internal circumference with the extension to the vessel circumference being detected by a pressure sensor. When the loop at the tip of the catheter is expanded to the size of the vessel lumen, the loop expansion stops and further expansion by a user or automatic motor drive is inhibited. The catheter device can be used as a marker for marking a position where a stent is to be deployed or for marking a position for valve placement or for deploying another device in a body cavity.

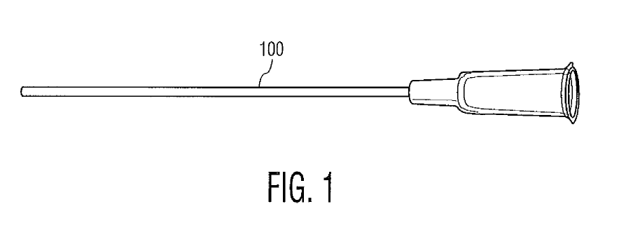

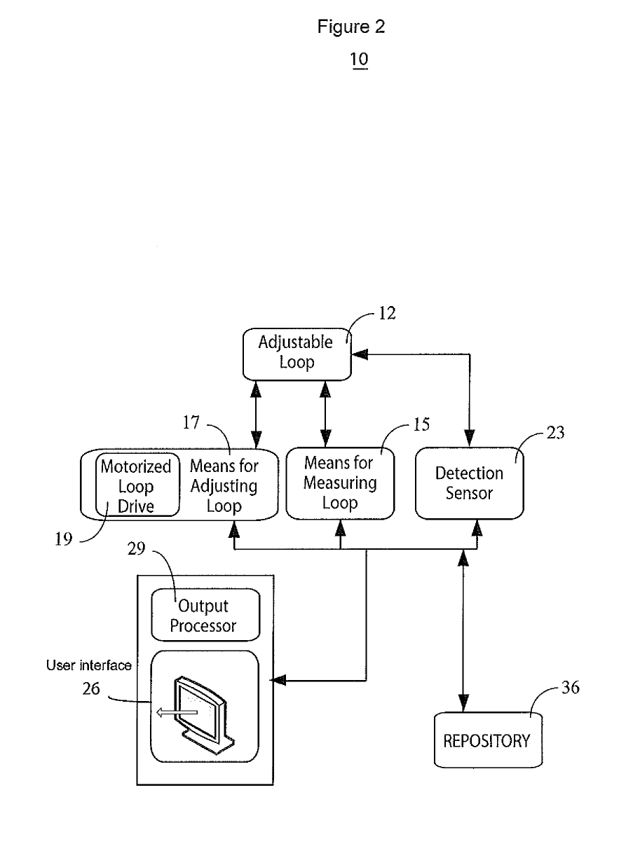

It is desirable to measure a vessel internal cross-sectional area or circumference with accuracy to determine degree of vessel stenosis and it is desirable to measure circumference of a non-circular vessel cross-section where stenosis is present or there is vessel distortion. FIG. 2 shows catheter device 10 for measuring vessel internal circumference. In contrast, FIG. 1 shows a known catheter 100 used in a hospital and that does not measure vessel internal circumference or cross-sectional area. Catheter device 10 comprises loop 12 having an adjustable circumference that is adjustable within a vessel. The loop material may comprise flexible metal wire of fine (or other) diameter, for example. The loop may alternatively comprise plastic, composite material, string or other flexible linear material that is extendable and conformable to a circular or non-circular cross-section of vessels of different internal diameter. The vessels of different internal diameter vary in dimension from relatively large diameter vessels of an Aorta, through medium size vessels of arteries and veins to fine diameter vessels comprising capillaries, for example.

Catheter device 10 includes a means for adjusting loop circumference length as well as shape (unit 17), to conform to vessel cross-section. The means for adjusting loop circumference unit 17 is motor 19 driven to automatically expand or retract the loop, in response to user command via user interface 26. The measurement and adjustment of loop size is performed by an electrical motor automatically until resistance is detected using a resistance detector. In another embodiment, the means for adjusting loop circumference unit 17 comprises a manually driven unit that expands or retracts loop 12 by using a rotational or screw type mechanism that may be similar to a screw type micrometer adjustment unit, for example. Alternative embodiments may use different extension and retraction mechanisms. Detection sensor 23 detects when loop 12 material is fully extended to the inner circumference of an anatomical vessel. Sensor 23 comprises a pressure sensor (e.g., a transducer) that detects resistance to further extension of loop 12. At least one repository 36 stores programmed instruction for execution by a processor controlling operation of the catheter system as well as other operational data including resistance thresholds, data supporting output and communication of measurement data for display or communication to other units, patient specific data, alert thresholds and loop adjustment characteristics. The resistance thresholds in one embodiment are patient specific and vary depend on patient demographic characteristics (age, weight, height, gender, for example). Loop adjustment characteristics include loop adjustment speed. Further, a movement sensor in unit 23 detects when catheter 10 is being moved (e.g., a catheter is being extracted) and automatically initiates minimization of loop 12 to facilitate catheter extraction.

A resistance level exceeding a predetermined threshold level is detected by sensor 23 and used to prevent further extension of loop 12 either by motor 19 or by manual adjustment. In response to a resistance level exceeding the predetermined threshold level being detected by sensor 23, unit 17 inhibits further motor 19 driven extension of loop 12 by disabling motor 19. Alternatively, in response to a resistance level exceeding the predetermined threshold level being detected by sensor 23, unit 17 inhibits further manual extension of loop 12 by a user via a disabling clutch mechanism preventing a user from advancing loop 12 material,

A means for measuring adjusted loop circumference (unit 15) dynamically measures circumference of loop 12 in response to extension and retraction of the loop. The measurement means 15 measures the circumference of loop 12 by detection of the length of loop 12 material extended. This is determined based on markings on loop 12 material detected by an infra-red or other reading means. Alternative measurement means may measure the circumference of loop 12 by detection of length of loop material remaining to be extended. Output unit 29 sends data indicating the measured loop circumference or cross-sectional area derived using the measured loop circumference for presentation to a user.

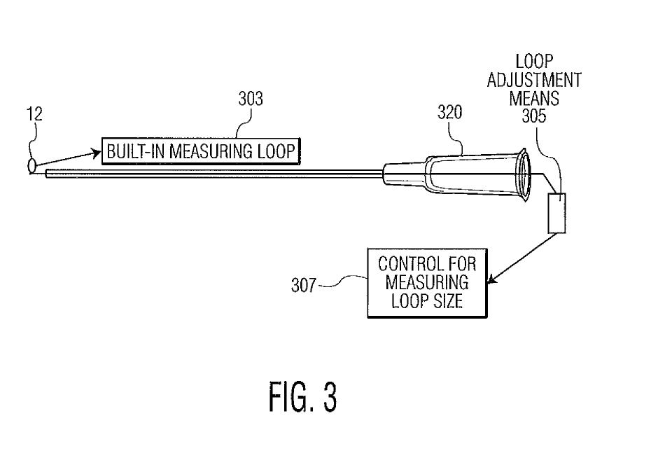

FIG. 3 shows a catheter system for measuring vessel internal circumference. Catheter body 320 houses loop 12 mounted at the invasive end of the catheter. Built in loop extension and retraction means 303 is controlled via a control unit 305 mounted at the external non-invasive end of the catheter. In response to insertion of the invasive end of the catheter in a vessel and initiation of vessel internal circumference measurement by a user via unit 305, the catheter initiates extension of loop 12 and determination of the loop circumference. Extension of loop 12 and measurement of loop 12 circumference is performed and controlled by unit 307 (e.g., including a processor) in response to commands entered by a user via unit 305. The catheter system provides direct measurement of vessel lumen size, valve size or size of other body cavities and minimizing error in measurement. A physician may also select a stent or valve deployment site by using the loop as a marker in X-ray imaging.

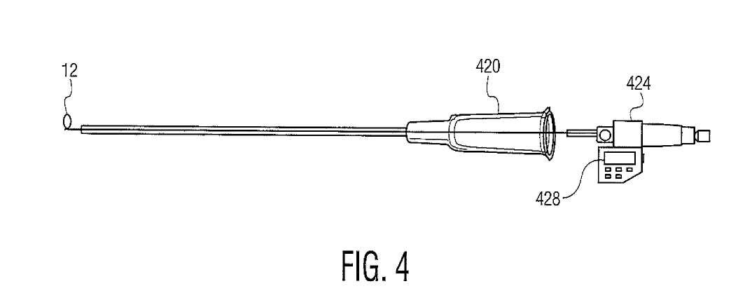

FIG. 4 shows catheter device 420 including automatic motorized unit 424 for controlling and performing measurement of vessel internal circumference in response to user commands entered via user interface 428. Catheter device 420 performs measurement of vessel internal circumference and also enables a user to mark a site within a patient vessel or other portion of patient anatomy. Loop 12 (FIG. 2) is made of X-ray opaque material and acts as a marker in an X-ray image. Loop 12 appears as a line in an image when the loop is perpendicular to the image and a physician is able to determine a precise image and catheter orientation facilitating valve alignment. The marked anatomical site facilitates user deployment of a stent or valve or any other invasive medical device or instrument, Further, catheter 420 advantageously enables measurement of a vessel lumen, a size of an anatomical cavity or size of a cardiac valve, for example. The catheter system provides a mechanical way to measure vessel stenosis and valves or cavities within a body. The system is usable to measure a body cavity such as a gastroesophageal sphincter or pyloric sphincter, for example.

In one embodiment, a catheter system employing a micrometer-like device, as previously described, determines loop 12 (FIG. 2) has expanded to the size of the lumen. In an embodiment, the catheter system includes a clutch that detects a preset resistance that stops further expansion of the loop. The catheter system in another embodiment makes a substantially circular estimate of lumen and valve cross-section from a determined circumference. The system is of particular use in heart (and other) valve opening measurements and positioning a medical device for valve replacement.

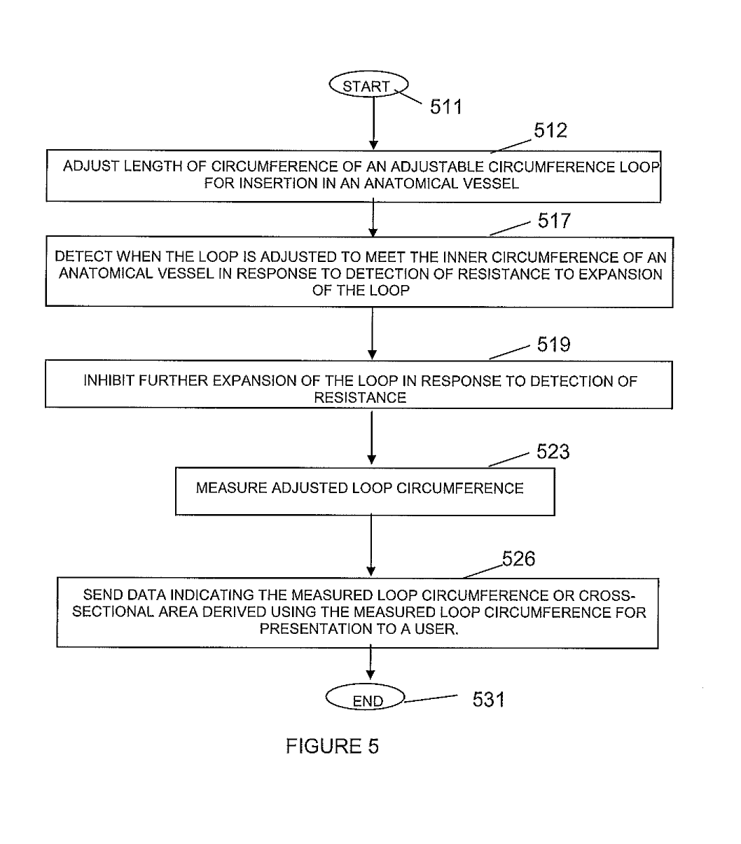

FIG. 5 shows a flowchart of a process used by a catheter system for measuring vessel internal circumference. In step 512, following the start at step 511, unit 17 (FIG. 2) adjusts length of circumference of an adjustable circumference loop 12 for use in an anatomical vessel, in response to a user command entered via user interface 26 initiating vessel lumen measurement. Unit 17 comprises a means for automatically expanding the loop and terminating loop expansion in response to detection the loop meets an inner surface of the anatomical vessel. In one embodiment, unit 17 comprises a motor 19 for driving loop extension and retraction in response to user command to initiate or terminate a vessel internal circumference measurement. In another embodiment, the means for adjusting the length of the circumference of the loop of unit 17 comprises a manual means for extending the loop and for retracting loop material.

The loop 12 material comprises at least one of (a) flexible metal wire, (b) plastic and (c) composite material, conformable to a circular or non-circular cross-section of a vessel. Detection sensor 23 in step 517, detects when the loop is adjusted to meet the inner circumference of an anatomical vessel in response to detection of resistance to expansion of the loop exceeding a predetermined threshold resistance. In one embodiment, the sensor is a pressure sensor comprising a transducer for providing an electrical signal in response to detected pressure. In step 519, unit 17 inhibits further expansion of the loop in response to detection of resistance. Unit 15 in step 523 measures adjusted loop circumference. The means for measuring adjusted loop circumference of unit 15 measures the circumference of the loop by detection of the length of loop material extended determined based on markings on loop material detected by an electromagnetic (e.g., infra-red) reading means. Other reading means may alternatively be used. Further, output processor 29 in step 526 sends data indicating the measured loop circumference or cross-sectional area derived using the measured loop circumference for presentation to a user. The process of FIG. 5 terminates at step 531.

A processor as used herein is a device for executing machine-readable instructions stored on a computer readable medium, for performing tasks and may comprise any one or combination of, hardware and firmware. A processor may also comprise memory storing machine-readable instructions executable for performing tasks. A processor acts upon information by manipulating, analyzing, modifying, converting or transmitting information for use by an executable procedure or an information device, and/or by routing the information to an output device. A processor may use or comprise the capabilities of a computer, controller or microprocessor, for example, and is conditioned using executable instructions to perform special purpose functions not performed by a general purpose computer. A processor may be coupled (electrically and/or as comprising executable components) with any other processor enabling interaction and/or communication there-between. A user interface processor or generator is a known element comprising electronic circuitry or software or a combination of both for generating display images or portions thereof. A user interface comprises one or more display images enabling user interaction with a processor or other device.

An executable application, as used herein, comprises code or machine readable instructions for conditioning the processor to implement predetermined functions, such as those of an operating system, a context data acquisition system or other information processing system, for example, in response to user command or input. An executable procedure is a segment of code or machine readable instruction, sub-routine, or other distinct section of code or portion of an executable application for performing one or more particular processes. These processes may include receiving input data and/or parameters, performing operations on received input data and/or performing functions in response to received input parameters, and providing resulting output data and/or parameters. A user interface (UI), as used herein, comprises one or more display images, generated by a user interface processor and enabling user interaction with a processor or other device and associated data acquisition and processing functions.

The UI also includes an executable procedure or executable application. The executable procedure or executable application conditions the user interface processor to generate signals representing the UI display images. These signals are supplied to a display device which displays the image for viewing by the user. The executable procedure or executable application further receives signals from user input devices, such as a keyboard, mouse, light pen, touch screen or any other means allowing a user to provide data to a processor. The processor, under control of an executable procedure or executable application, manipulates the UI display images in response to signals received from the input devices. In this way, the user interacts with the display image using the input devices, enabling user interaction with the processor or other device. The functions and process steps herein may be performed automatically or wholly or partially in response to user command. An activity (including a step) performed automatically is performed in response to executable instruction or device operation without user direct initiation of the activity.

The system and processes of FIGS. 2-5 are not exclusive. Other systems, processes and menus may be derived in accordance with the principles of the invention to accomplish the same objectives. Although this invention has been described with reference to particular embodiments, it is to be understood that the embodiments and variations shown and described herein are for illustration purposes only. Modifications to the current design may be implemented by those skilled in the art, without departing from the scope of the invention. A catheter system automatically measures vessel internal circumference using a measuring loop of adjustable circumference at the tip of the catheter that may also be used for marking a position where a stent is to be deployed or for marking a position for valve placement or for deploying another device in a body cavity. Further, the processes and applications may, in alternative embodiments, be located on one or more (e.g., distributed) processing devices on a network linking the units of FIG. 2. Any of the functions and steps provided in FIGS. 2-5 may be implemented in hardware, software or a combination of both.

What is claimed is:

1. A catheter system, comprising:
a catheter device including,
a loop having an adjustable length circumference, said length being adjustable within an anatomical vessel,
a means for adjusting the length of the circumference of said loop and
a means for measuring adjusted loop circumference; and
an output unit for sending data indicating the measured loop circumference or cross-sectional area derived using said measured loop circumference for presentation to a user.

2. A system according to claim 1, including
a means for detecting when said loop is adjusted to meet the inner circumference of an anatomical vessel in response to detection of resistance to expansion of said loop.

3. A system according to claim 2, including
a means for automatically expanding said loop and terminating loop expansion in response to detection said loop meets an inner surface of said anatomical vessel.

4. A system according to claim 2, including
a pressure sensor for detecting resistance to expansion of said loop.

5. A system according to claim 4, wherein
said pressure sensor comprises a transducer for providing an electrical signal in response to detected pressure.

6. A system according to claim 1, wherein
said means for adjusting the length of the circumference of said loop comprises a motor for driving loop extension and retraction in response to user command to initiate or terminate a vessel internal circumference measurement.

7. A system according to claim 1, wherein
said means for measuring adjusted loop circumference measures the circumference of said loop by detection of the length of loop material extended determined based on markings on loop material.

8. A system according to claim 7, wherein
said means for measuring adjusted loop circumference measures length of loop material extended in response to measurement determined based on markings on loop material detected by an electromagnetic reading means.

9. A system according to claim 1, wherein
said means for adjusting the length of the circumference of said loop comprises a manual means for extending said loop.

10. A system according to claim 9, wherein
said means for adjusting the length of the circumference of said loop comprises a manual means for retracting loop material.

11. A system according to claim 1, wherein
said loop material comprises at least one of, (a) flexible metal wire, (b) plastic and (c) composite material, conformable to a circular or non-circular cross-section of a vessel.

12. A catheter system, comprising:
a catheter device including,
a loop having an adjustable length circumference, said length being adjustable within an anatomical vessel,
a means for adjusting the length of the circumference of said loop,
a pressure sensor for detecting resistance to expansion of said loop and inhibiting further expansion of said loop in response to detection of resistance, and
a means for measuring adjusted loop circumference; and
an output unit for sending data indicating the measured loop circumference or cross-sectional area derived using said measured loop circumference for presentation to a user.

13. A system according to claim 12, including
said pressure sensor detects resistance to expansion of said loop in response to resistance exceeding a predetermined threshold resistance.

14. A system according to claim 12, wherein
said means for adjusting the length of the circumference of said loop comprises a motor for driving loop extension and retraction in response to user command to initiate or terminate a vessel internal circumference measurement,

15. A method employed by a catheter system for determining vessel lumen size, comprising the activities of:
adjusting length of circumference of an adjustable circumference loop for use in an anatomical vessel;
inhibiting further expansion of said loop in response to detection of resistance;
measuring adjusted loop circumference; and
sending data indicating the measured loop circumference or cross-sectional area derived using said measured loop circumference for presentation to a user.
